(19) United States
(12) Patent Application Publication
(10) Pub. No.: US 2025/0145760 A1
(43) Pub. Date: May 8, 2025

(54) EPOXY RESIN COMPOSITIONS COMPRISING DICYANDIAMID CURING AGENT

(71) Applicant: 3M INNOVATIVE PROPERTIES COMPANY, St. Paul, MN (US)

(72) Inventor: Christian Heering, Duesseldorf (DE)

(21) Appl. No.: 18/834,100

(22) PCT Filed: Feb. 9, 2023
(86) PCT No.: PCT/IB2023/051183

§ 371 Date: Jul. 29, 2024

Related U.S. Application Data
(60) Provisional application No. 63/315,160, filed on Mar. 1, 2022.

(51) Int. Cl. C08G 59/40; C07C 279/28; C08G 59/24; C08G 59/50; C09J 5/00; C09J 163/00

(52) U.S. Cl. CPC C08G 59/4064; C07C 279/28; C08G 59/245; C08G 59/4071; C08G 59/504; C09J 5/00; C09J 163/00; C09J 2463/00

(57) ABSTRACT

A composition is described comprising an epoxy resin and a dicyandiamide salt comprising an anion selected from sulfonate or phosphonate. In typical embodiments, the dicyandiamide cation has the formula: wherein one or more of R1, R2, R3, and R4 are hydrogen. Also described is a method of bonding, an adhesive bonded article, and a method of synthesizing a dicyandiamide salt.

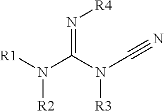

### SUMMARY

Although various epoxy resin curatives have been described, industry would find advantage in curatives that are amenable to curing at lower temperatures.

In one embodiment, a composition an epoxy resin a dicyandiamide salt comprising an anion selected from sulfonate or phosphonate.

In typical embodiments, the dicyandiamide cation has the formula:

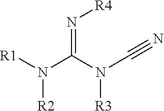

wherein one or more of R1, R2, R3, and R4 are hydrogen and optionally at least one of R1, R2, R3, and R4 is alkyl, aryl, alkylaryl, or arylalkyl.

In typical embodiments, the anion has the formula:

RSO₃⁻ or RPO₃⁻²

wherein R is an aliphatic or aromatic organic group.

In some embodiments, the composition is suitable for use as an adhesive.

In another embodiment, a method of bonding is described comprising utilizing the composition of the previous claims to bond a first substrate to a second substrate.

In another embodiment, an adhesive bonded article is described comprising a first and second substrate bonded with the composition of the previous claims.

In another embodiment, a method of synthesizing a dicyandiamide compound is described comprising forming an aqueous solution of dicyandiamide in deionized water: adding a sulfonic or phosphonic acid to the aqueous solution; and removing the water.

### DETAILED DESCRIPTION

Presently described are compositions comprising an epoxy resin and a dicyandiamide epoxy resin curative. The epoxy resin curative comprises a dicyandiamide cation and an anion selected from sulfonate or phosphonate.

Dicyandiamide (DICY or DCD), also known as cyanoguanidine, is the dimer for cyanamide or for cyanoguanidine. Dicyandiamide is white crystalline powder with the molecular formula of C₂H₄N₄ and CAS number 461-58-5. A dicyandiamide cation can be represented by the following formula:

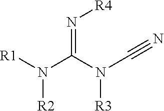

wherein one or more of R1, R2, R3, and R4 are hydrogen and optionally at least one of R1, R2, R3, and R4 is alkyl (e.g. C1-C12), aryl (e.g. phenyl), (e.g. C1-C12) alkylaryl, or (e.g. C1-C12) arylalkyl.

When one or more of R1, R2, R3, and R4 are hydrogen, the dicyandiamide structure has a positive charge. In typical, embodiments, R1 and/or R2 are hydrogen. In some embodiments, R1, R2, R3, and R4 are hydrogen. In other embodiments, R1, R3, and R4 are hydrogen and R2 is alkyl (e.g. C1-C12), aryl (e.g. phenyl), (e.g. C1-C12) alkylaryl, or (e.g. C1-C12) arylalkyl.

Examples of substituted dicyandiamides include for example N-cyano-N′-methylguanidine, 1,3-dimethyl-2-cyanoguanidine, N′-cyano-N,N-dimethylguanidine, N-cyano-N′-ethylguanidine, N-cyano-N′-ethyl-N″-methylguanidine, N-cyano-N′-phenylguanidine, N-cyano-N′-cyclopropylguanidine, N-cyano-N′-cyclohexylguanidine.

The epoxy resin curative comprising a dicyandiamide cation and an anion selected from sulfonate or phosphonate can be synthesized by any suitable method. The epoxy resin curatives are typically prepared by reacting dicyandiamide with an acid such as sulfonic acid or phosphonic acid.

Suitable acids are represented by the formula:

R[SO₃]ₙH

R[PO₃]ₙH₂
wherein n is 1 or 2; and
R is an aliphatic or aromatic organic group.

Typical aliphatic organic groups include alkyl optionally comprising heteroatoms such as oxygen or nitrogen. The aliphatic (e.g. alkyl) organic groups typically comprise 1 to 18 carbon atoms. In some embodiments, the aliphatic (e.g. alkyl) organic groups comprise no greater than 12 carbon atoms. In other embodiments, the aliphatic (e.g. alkyl) organic groups comprise no greater than 6 or 4 carbon atoms.

In some embodiments, R is a C1, C2, C3, or C4 alkyl group optionally substituted with halogen (e.g. F) or hydroxy.

In other embodiments, R is an amine group (e.g. NH₂) or a C₁-C₄ alkyl amine.

Typical aromatic organic groups include benzyl, toluene, phenyl, diphenyl, and naphthyl optionally substituted for example with C₁-C₄ alkyl (e.g. methyl), halogen, or nitrogen. In some embodiments, the aromatic organic group further comprises an alkylene group or in other words is an alkylaryl group such as in the case of alkylbenzene sulfonic acids. The aromatic organic groups typically comprise 6 to 18 carbon atoms. The alkylene group of the alkylaryl group may comprise 1 to 18 carbon atoms (e.g. ethylene)

Some representative acids are described in the following Table A.

TABLE A
Acids

| | |
|---|---|
| TSA | 4-Methylbenzene-1-sulfonic acid monohydrate, |
| | p-Toluenesulfonic acid monohydrate, |
| | obtained from Sigma Aldrich, St. Louis, MO, USA |
| | 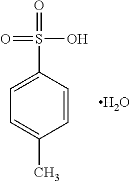 |
| MSA | Methylsulfonic acid, obtained from Sigma Aldrich, St. Louis, MO, |
| | USA |
| | 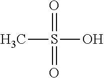 |
| BSA | Benzenesulfonic acid monohydrate, obtained from Sigma Aldrich |
| | 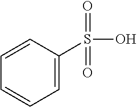 |
| TAU | Taurine, 2-Aminoethane-1-sulfonic acid, obtained from Sigma |
| | Aldrich |
| | 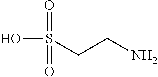 |
| ASA | Sulfamic acid, aminosulfonic acid, obtained from Sigma Aldrich |
| | 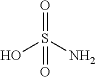 |
| MPA | Methylphosphonic acid, obtained from Sigma Aldrich |
| | 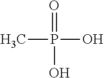 |
| BPA | Benzenephosphonic acid, obtained from Sigma Aldrich |
| | 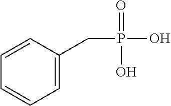 [SMILES: O=P(O)(O)Cc1ccccc1] |

Other suitable aliphatic sulfonic acids include for example trifluoromethane sulfonic acid, ethane sulfonic acid, propane sulfonic acid, 3-hydroxy propane sulfonic acid.

Other suitable aromatic sulfonic acids include for example benzenedisulfonic acid, o- and p-toluenesulfonic acid, toluene disulfonic acid, C₂-C₁₈ alkylbenzenesulfonic acids, naphthylenesulfonic acid, and C₁-C₁₈ alkylnaphthalenedisulfonic acids.

Other suitable aliphatic phosphonic acids include for example ethane phosphonic acid, propane phosphonic acid, octyl phosphonic acid, decyl phosphonic acid, hexadecyl phosphonic acid, 3-amine propane phosphonic acid.

Other suitable aromatic phosphonic acids include for example phenyl phosphonic acid, 4-bromophenyl phosphonic acid, 4-chlorophenyl phosphonic acid, 4-nitrophenyl phosphonic acid, diphenyl-4-phosphonic acid.

In some embodiments, the reaction of dicyandiamide with sulfonic acid is conducted in a suitable solvent, as described in U.S. Pat. No. 2,433,394. Suitable solvent includes for example ethers, esters, ketones, and acids. In another embodiment, dicyandiamide can be reacted directly with certain sulfonates at moderately higher temperatures are described in U.S. Pat. No. 2,473,112.

In some favored embodiments, the dicyandiamide salts are synthesized in an aqueous solution. The method of synthesizing a dicyandiamide salt generally comprises forming an aqueous solution of the dicyandiamide compound in deionized water: adding a sulfonic or phosphonic acid to the aqueous solution; and removing the water.

The concentration of dicyandiamide compound in the aqueous solution typically ranges from 1.0 g/L to 25 g/L. In some embodiments, an equimolar amount of acid it typically added. Alternatively, the amount of acid may be less than an equimolar amount. In this embodiment, the reaction product predominantly comprises the dicyandiamide salt in combination with a minor amount of dicyandiamide compound. The step of removing the water can be accomplished by various ordinary means known in the art such as rotary evaporation. The resulting dicyandiamide (e.g. sulfonate or phosphonate) salt can be a solid or liquid at 25° C. In some embodiments, the dicyandiamide salt can be dried (e.g. in an oven at 80° C. for at least 2 h).

One representative reaction scheme is as follows:

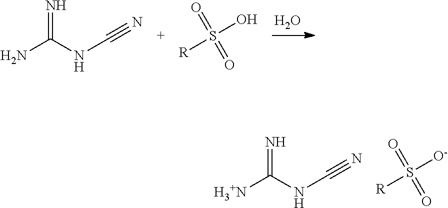

The dicyandiamide salt is suitable for use as an epoxy resin curative.

In some embodiments, the dicyandiamide salt is utilized as an accelerator in combination with at least one other (i.e. different) epoxy resin curing agent. Common classes of curatives for epoxy resins include amines, amides, ureas, imidazoles, and thiols. The curing agent is typically highly reactive with the epoxide groups at ambient temperature.

In some embodiments, the curing agent in an amine curing agent that comprises reactive —NH groups or reactive —NR₁R₂ groups wherein R₁ and R₂ are independently H or C₁ to C₄ alkyl, and most typically H or methyl.

One class of curing agents are primary, secondary, and tertiary polyamines. The polyamine curing agent may be straight-chain, branched, or cyclic. In some favored embodiments, the polyamine crosslinker is aliphatic. Alternatively, aromatic polyamines can be utilized.

Useful polyamines are of the general formula R₅—(NR₁R₂)ₓ wherein R₁ and R₂ are independently H or alkyl, R₅ is a polyvalent alkylene or arylene, and x is at least two. The alkyl groups of R₁ and R₂ are typically C₁ to C₁₈ alkyl, more typically C₁ to C₄ alkyl, and most typically methyl. R₁ and R₂ may be taken together to form a cyclic amine. In some embodiment x is two (i.e. diamine). In other embodiments, x is 3 (i.e. triamine). In yet other embodiments, x is 4.

Useful diamines may be represented by the general formula:

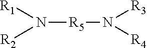

wherein R₁, R₂, R₃ and R₄ are independently H or alkyl, and Rs is a divalent alkylene or arylene. In some embodiments, R₁, R₂, R₃ and R₄ are each H and the diamine is a primary amine. In other embodiments, R₁ and R₄ are each H and R₂, and R₄ are each independently alkyl; and the diamine is a secondary amine. In yet other embodiments, R₁, R₂, R₃ and R₄ are independently alkyl and the diamine is a tertiary amine.

In some embodiments, primary amines are preferred. Examples include hexamethylene diamine: 1,10-diaminodecane: 1,12-diaminododecane: 2-(4-aminophenyl) ethylamine: isophorone diamine; norbornane diamine 4,4′-diaminodicyclohexylmethane; and 1,3-bis(aminomethyl)cyclohexane. Illustrative six member ring diamines include for example piperazine and 1,4-diazabicyclo[2.2.2]octane (“DABCO”).

Other useful polyamines include polyamines having at least three amino groups, wherein the three amino groups are primary, secondary, or a combination thereof. Examples include 3,3′-diaminobenzidine and hexamethylene triamine.

Common curing agents used to cure cycloaliphatic epoxy resin include anhydrides derived from a carboxylic acid which possesses at least one anhydride group. Such anhydride curing agents are described in U.S. Pat. No. 6,194,024: incorporated herein by reference.

In some embodiments, the other epoxy curing agent may be dicyandiamide.

The concentration of the curing agent(s) is typically less than about 3, 2.5, 2, 1.5 or 1 wt.-%, based on the weight of the total epoxy resin composition. In some embodiments, the amount of curing agent is at least 0.005, 0.01, 0.025, 0.05, 0.1, 0.2, 0.3, 0.4, or 0.5 wt.-%.

### Epoxy Resin

The epoxy resins or epoxides that are useful in the composition may be any organic compound having at least one oxirane ring that is polymerizable by ring opening, i.e., an average epoxy functionality greater than one, and preferably at least two. The epoxides can be monomeric or polymeric, and aliphatic, cycloaliphatic, heterocyclic, aromatic, hydrogenated, or mixtures thereof. Preferred epoxides contain more than 1.5 epoxy group per molecule and preferably at least 2 epoxy groups per molecule. The useful materials typically have a weight average molecular weight of about 150 to about 10,000, and more typically of about 180 to about 1,000. The molecular weight of the epoxy resin is usually selected to provide the desired properties of the cured composition. Suitable epoxy resins include linear polymeric epoxides having terminal epoxy groups (e.g., a diglycidyl ether of a polyoxyalkylene glycol), polymeric epoxides having skeletal epoxy groups (e.g., polybutadiene poly epoxy), and polymeric epoxides having pendent epoxy groups (e.g., a glycidyl methacrylate polymer or copolymer), and mixtures thereof. The epoxide-containing materials include compounds having the general formula:

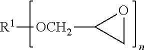

where R1 is an alkyl, alkyl ether, or aryl, and n is 1 to 6.

These epoxy resins include aromatic glycidyl ethers, e.g., such as those prepared by reacting a polyhydric phenol with an excess of epichlorohydrin, cycloaliphatic glycidyl ethers, hydrogenated glycidyl ethers, and mixtures thereof. Such polyhydric phenols may include resorcinol, catechol, hydroquinone, and the polynuclear phenols such as p,p′-dihydroxydibenzyl, p,p′-dihydroxydiphenyl, p,p′-dihydroxyphenyl sulfone, p,p′-dihydroxybenzophenone, 2,2′-dihydroxy-1, 1-dinaphthylmethane, and the 2,2′, 2,3′, 2,4′, 3,3′, 3,4′, and 4,4′ isomers of dihydroxydiphenylmethane, dihydroxydiphenyldimethylmethane, dihydroxydiphenylethylmethylmethane, dihydroxydiphenylmethylpropylmethane, dihydroxydiphenylethylphenylmethane, dihydroxydiphenylpropylphenylmethane, dihydroxydiphenylbutylphenylmethane, dihydroxydiphenyltolylethane, dihydroxydiphenyltolylmethylmethane, dihydroxydiphenyldicyclohexylmethane, and dihydroxydiphenylcyclohexane.

Also useful are polyhydric phenolic formaldehyde condensation products as well as polyglycidyl ethers that contain as reactive groups only epoxy groups or hydroxy groups. Useful curable epoxy resins are also described in various publications including, for example, “Handbook of Epoxy Resins” by Lee and Nevill, McGraw-Hill Book Co., New York (1967), and Encyclopedia of Polymer Science and Technology, 6, p. 322 (1986).

The choice of the epoxy resin used depends upon the end use for which it is intended. Epoxides with flexibilized backbones may be desired where a greater amount of ductility is needed in the bond line. In some embodiments, the composition is suitable for use as a structural adhesive. Materials such as diglycidyl ethers of bisphenol A and diglycidyl ethers of bisphenol F can provide desirable structural adhesive properties that these materials attain upon curing, while hydrogenated versions of these epoxies may be useful for compatibility with substrates having oily surfaces.

Examples of commercially available epoxides useful in the present disclosure include diglycidyl ethers of bisphenol A (e.g. those available under the trade designations EPON 828, EPON 1001, EPON 1004, EPON 2004, EPON 1510, and EPON 1310 from Momentive Specialty Chemicals, Inc., and those under the trade designations D.E.R. 331, D.E.R. 332, D.E.R. 334, and D.E.N. 439 available from Dow Chemical Co.): diglycidyl ethers of bisphenol F (e.g., that are available under the trade designation ARALDITE GY 281 available from Huntsman Corporation): silicone resins containing diglycidyl epoxy functionality: flame retardant epoxy resins (e.g., that are available under the trade designation DER 560, a brominated bisphenol type epoxy resin available from Dow Chemical Co.); and 1,4-butanediol diglycidyl ethers.

Epoxy-containing compounds having at least one glycidyl ether terminal portion, and preferably, a saturated or unsaturated cyclic backbone may optionally be added to the composition as reactive diluents. Reactive diluents may be added for various purposes such as to aid in processing, e.g., to control the viscosity in the composition as well as during curing, to flexibilize the cured composition, and to compatibilize materials in the composition.

Examples of such diluents include: diglycidyl ether of cyclohexanedimethanol, diglycidyl ether of resorcinol, p-tert-butyl phenyl glycidyl ether, cresyl glycidyl ether, diglycidyl ether of neopentyl glycol, triglycidyl ether of trimethylolethane, triglycidyl ether of trimethylolpropane, triglycidyl p-amino phenol, N,N′-diglycidylaniline, N,N,N′N′-tetraglycidyl meta-xylylene diamine, and vegetable oil polyglycidyl ether. Reactive diluents are commercially available under the trade designation HELOXY 107 and CARDURA N10 from Momentive Specialty Chemicals, Inc. The composition may contain a toughening agent to aid in providing the desired overlap shear, peel resistance, and impact strength.

The (e.g. adhesive) composition desirably contains one or more epoxy resins having an epoxy equivalent weight of at least 100, 200 or 300 and typically no greater than 1500, 1200, or 1000. In some embodiments, the adhesive contains two or more epoxy resins, wherein at least one epoxy resin has an epoxy equivalent weight of from about 300 to about 500, and at least one epoxy resin has an epoxy equivalent weight of from about 1000 to about 1200.

In some embodiments, the (e.g. structural) adhesive composition comprises one or more epoxy resins in an amount of at least 20, 25 or 30 wt. % and typically no greater than 95, 90, 85, 80, 75, or 70 wt. % of the unfilled (e.g. adhesive) composition or in other words the total amount of organic components except for organic polymeric fillers.

The epoxy resin composition may optionally comprise other components, as known in the art, including for examples film-forming polymers, such as (meth)acrylic) polymers and low molecular weight, liquid (at 25° C.) hydroxy-functional polyol. Polyols can be suitable for retarding the curing reaction so that the “open time” of the adhesive composition can be increased.

The composition may optionally further comprise various additives such as fillers, stabilizers, plasticizers, tackifiers, flow control agents, cure rate retarders, adhesion promoters (for example, silanes and titanates), adjuvants, impact modifiers, and the like, such as silica, glass, clay, talc, pigments, colorants, glass beads or bubbles, and antioxidants.

In some embodiments, the compositions described herein may be provided as two-part compositions that can be utilized with cartridges as well as dispensing and mixing apparatus that are currently used for two-part epoxy compositions. The epoxy resin is provided in a first part. The second part comprises an epoxy (e.g. amine) curing agent. The dicyandiamide salt may be provided in either part, but is most typically included in the second part. The first and second part are combined when dispensing the epoxy resin composition.

In some embodiment, the composition has an onset temperature of an exotherm as measured by Differential Scanning calorimetry of less than 180, 175, 170, 165, 160, 155, 150, or 145° C. In some embodiments, the composition has a peak temperature of an exotherm as measured by Differential Scanning calorimetry of less than 190, 185, 180, or 170° C. The composition can have a lower onset or peak temperature as compared to the same composition without the dicyandiamide salt.

For example, as depicted in the forthcoming examples the onset temperature of the dicyandiamide salt in combination with an amine curing agent (e.g. dicyandiamide) can be less than dicyandiamide alone.

The presence of the dicyandiamide salt can reduce the onset or peak temperature by at least 5, 10, 15, 20, 35, 30, 25, or 40° C.

In some embodiments, the composition described herein is suitable for use as a structural or semi-structural adhesive.

“Semi-structural adhesives” are those cured adhesives that have an overlap shear strength (according to the test method of the examples) of at least about 0.5 MPa, more preferably at least about 1.0 MPa, and most preferably at least about 1.5 MPa. Those cured adhesives having particularly high overlap shear strength, however, are referred to as structural adhesives. “Structural adhesives” are those cured adhesives that have an overlap shear strength of at least about 3, 4, 5, 6, or 7 MPa. In some embodiments, the overlap shear strength is no greater than about 12 MPa. The overlap shear strength can be tested according to the test method described in the examples.

In one embodiment, a method of bonding is described comprising utilizing the composition of the previous claims to bond a first substrate to a second substrate. The curing of the epoxy resin occurs after applying the (e.g. adhesive) composition between the substrates. The first and second substrate may be the same substrates or different substrates.

The (e.g. adhesive) composition may be coated upon a variety of flexible and inflexible substrates. Examples include for example plastic films such as polyolefins (e.g. polypropylene, polyethylene), polyvinyl chloride, polyester (polyethylene terephthalate), polycarbonate, polymethyl(meth)acrylate (PMMA), cellulose acetate, cellulose triacetate, and ethyl cellulose. In some embodiments, the substrate is comprised of a bio-based material such as polylactic acid (PLA).

Substrates may also be prepared of fabric such as woven fabric formed of synthetic or natural fibrous materials such as cotton, nylon, rayon, glass, ceramic materials, and the like or nonwoven fabric such as air laid webs of natural or synthetic fibers or blends of these.

The substrate may also be formed of metal (e.g. steel, aluminum, copper), metalized polymer films, ceramic sheet materials, or foam (e.g., polyacrylic, polyethylene, polyurethane, neoprene), and the like.

In another embodiment, an adhesive bonded article is described comprising a first and second substrate bonded with the epoxy resin composition described herein.

### EXAMPLES

Unless otherwise noted or readily apparent from the context, all parts, percentages, ratios, etc. in the Examples and the rest of the specification are by weight.

TABLE 1
Materials Used in the Examples

| Abbreviation | Description and Source |
|---|---|
| DICY | Dicyandiamide, cyanoguanidine, obtained from Huntsman, |
| | The Woodlands, TX, USA |
| DICY-acid | Accelerator based on a DICY and an acid |
| TSA | 4-Methylbenzene-1-sulfonic acid monohydrate, p- |
| | Toluenesulfonic acid monohydrate, obtained from Sigma |
| | Aldrich, St. Louis, MO, USA |
| MSA | Methylsulfonic acid, obtained from Sigma Aldrich, St. |
| | Louis, MO, USA |
| BSA | Benzenesulfonic acid monohydrate, obtained from Sigma |
| | Aldrich |
| TAU | Taurine, 2-Aminoethane-1-sulfonic acid, obtained from |
| | Sigma Aldrich |
| ASA | Sulfamic acid, aminosulfonic acid, obtained from Sigma |
| | Aldrich |
| MPA | Methylphosphonic acid, obtained from Sigma Aldrich |
| BPA | Benzenephosphonic acid, obtained from Sigma Aldrich |
| DGEBA | Bisphenol A diglycidyl ether, Epikote 828, Hexion, |
| | Columbus, OH, USA |

### Test Methods

### Differential Scanning Calorimetry (DSC) Measurements According to DIN 65467.

The curable compositions were prepared by weighing the epoxy resin (DGEBA), adding the curative (DICY) and finally adding the accelerator (Examples). After a homogeneous mixture was achieved, the formulations were applied to the surface of the test panel for further testing in the manner specified below.

The DSC measurements were conducted on a Netzsch DSC 214 Polyma Differential Scanning calorimeter (commercially available from Netzsch GmbH). Formulation samples for measurements had a mass ranging from 10-20 mg. A heating rate of 5 Kmin⁻¹ was used at a temperature ranging from −20° C. to 300° C. The DSC onset temperature was calculated for a single peak with the peak temperature determined at the peak maximum. The DSC onset temperature serves as an indicator of the curing performance of the thermal curing system used in the exemplary curable compositions. The DSC onset temperatures are reported in ° C.

### Overlap Shear Strength (OLS) According to DIN EN 1465

The surface of the OLS metal sheets (steel, grade DX54+ZMB-RL1615) were cleaned with n-heptane. The metal sheets are left at ambient room temperature (23° C.+/−2° C., 50% relative humidity+/−5%) for 24 hours prior to testing and the OLS strength is measured as described below.

Overlap shear strength was determined according to DIN EN 1465 using a Zwick Z050 tensile tester (commercially available by Zwick GmbH & Co. KG, Ulm, Germany) operating at a cross head speed of 10 mm/min. For the preparation of an Overlap Shear Strength test assembly, a curable composition is placed onto one surface of a prepared metal sheet. Afterwards, the sample was covered by a second metal sheet forming an overlap joint of 10 mm. The overlap joints are then clamped together using two binder clips and the test assemblies are further stored at room temperature for 4 hours after bonding, and then placed into an air circulating oven from Heraeus for 30 minutes at 180° C. The next day, the samples are tested directly. Three samples are measured for each of the examples and results averaged and reported in MPa.

### Comparative Example

CEI was prepared by adding 0.5 g DICY to 5.0 g DGEBA and creating a homogeneous mixture.

### Examples

All combinations of cyanoguanidine with a corresponding acid (DICY-acid) were prepared according to the following procedure:

1.0 g of cyanoguanidine (84.08 g/mol, 1.18 10⁻³ mol) was dissolved in 20 mL of DI water using a round bottom flask. An equimolar amount of acid (1.18 10⁻³ mol) dissolved in 20 mL DI water was then added to the former and the combined solutions stirred for one hour at ambient conditions using a magnetic stirrer.

The water was then removed by rotary evaporation at lowest possible pressure for one hour at 60° C. Either a solid or clear liquid was obtained, which was further dried in an oven at 80° C. for at least 2 h to give the desired product.

Curable compositions were formulated with DGEBA, DICY and DICY-acid as described in following Error! Reference source not found. Error! Reference source not found.

Error! Reference source not found. Error! Reference source not found.

| | Formulation, amount in g | | | |
|---|---|---|---|---|
| Examples | DGEBA | DICY | DICY-acid | Acid |
| CE1 | 5 | 0.5 | — | — |
| 1 | 5 | 0.5 | 0.5 | MSA |
| 2 | 5 | 0.5 | 0.5 | TSA |
| 3 | 5 | 0.5 | 0.5 | BSA |
| 4 | 5 | 0.5 | 0.5 | TAU |
| 5 | 5 | 0.5 | 0.5 | ASA |
| 6 | 5 | 0.5 | 0.5 | MPA |
| 7 | 5 | 0.5 | 0.5 | BPA |

TABLE 2
DSC Test Results

| Examples | Tₒₙₛₑₜ, ° C. | Tₚₑₐₖ ° C. |
|---|---|---|
| CE1 | 184.0 | 194.1 |
| 1 | 146.4 | 200.3 |
| 2 | 158.9 | 183.0 |
| 3 | 151.6 | 169.3 |
| 4 | 175.2 | 186.1 |
| 5 | 174.1 | 188.1 |
| 6 | 156.9 | 187.7 |
| 7 | 159.4 | 173.0 |

TABLE 3
OLS Test Results (cured at 180° C. for 30 min)

| Examples | OLS in MPa |
|---|---|
| CE1 | 16.0 |
| 1 | 9.0 |
| 2 | 8.8 |
| 3 | 5.9 |
| 4 | 11.1 |
| 5 | 10.3 |
| 6 | 1.5 |
| 7 | 3.0 |

1. A composition comprising:
an epoxy resin; and
a dicyandiamide salt comprising an anion selected from sulfonate or phosphonate.

2. The composition of claim 1 wherein the dicyandiamide salt has a cation having the formula: 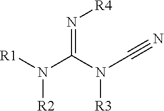
wherein one or more of R1, R2, R3, and R4 are hydrogen and optionally at least one of R1, R2, R3, and R4 is alkyl, aryl, alkylaryl, or arylalkyl.

3. The composition of claim 1 wherein the anion has the formula:
RSO₃⁻
wherein R is an aliphatic or aromatic organic group.

4. The composition of claim 1 wherein the anion has the formula:
RPO₃⁻²
wherein R is an aliphatic or aromatic organic group.

5. The composition of claim 1 wherein the composition further comprises an epoxy resin curing agent comprising amine groups.

6. The composition of claim 1 wherein the epoxy resin is provided in a first part; and
the dicyandiamide salt and an amine curing agent are provided in a second part.

7. The composition of claim 1 wherein the composition has an onset temperature of an exotherm as measured by Differential Scanning calorimetry of less than 180, 175, 170, 165, 160, 155, 150, or 145° C.

8. The composition of claim 1 wherein the composition has a peak temperature of an exotherm as measured by Differential Scanning calorimetry of less than 190, 185, 180, or 170° C.

9. The composition of claim 1 wherein the composition has an onset temperature of an exotherm as measured by Differential Scanning calorimetry that is lower than the same composition without the dicyandiamide salt.

10. The composition of claim 9 wherein presence of the dicyandiamide salt reduces the onset temperature by at least 5, 10, 15, 20, 35, 30, 25, or 40° C.

11. The composition of claim 1 wherein the composition is an adhesive.

12. The composition of claim 11 wherein the composition has an overlap shear of at least 1, 2, or 3 MPa.

13. A method of bonding comprising utilizing the composition of claim 1 to bond a first substrate to a second substrate.

14. A adhesive bonded article comprising a first and second substrate bonded with the composition of claim 1.

15. A method of synthesizing a dicyandiamide salt comprising:
forming an aqueous solution of dicyandiamide in deionized water;
adding a sulfonic or phosphonic acid to the aqueous solution; and
removing the water.
